(19) United States
(12) Patent Application Publication
(10) Pub. No.: US 2005/0064589 A1
(43) Pub. Date: Mar. 24, 2005

(54) Blastocyst culture media

(76) Inventor: Paul Kaplan, New City, NY (US)

Correspondence Address: OSTROLENK FABER GERB & SOFFEN, 1180 AVENUE OF THE AMERICAS, NEW YORK, NY, 100368403, US

(21) Appl. No.: 10/890,698

(22) Filed: Jul. 14, 2004

Related U.S. Application Data
(60) Provisional application No. 60/504,054, filed on Sep. 19, 2003.

(51) Int. Cl. C12N 5/08; C12N 5/02

(52) U.S. Cl. 435/366; 435/404

(57) ABSTRACT

A sequential human embryo culture system including a plurality of culture media corresponding to a plurality of patient and embryo groups, each medium including substances for supporting a human embryo. The culture media may include a buffer system for maintaining pH substantially constant during embryo development. The culture media may be substantially phosphate-free. The supporting substances may include sodium lactate and/or EDTA. In a method according to the invention, one of the sequential human embryo culture systems is used for supporting a human embryo.

### CROSS-REFERENCE TO RELATED APPLICATIONS

This application is based upon and claims priority of U.S. provisional application Ser. No. 60/504,054, filed Sep. 19, 2003, the disclosure of which is incorporated by reference herein.

### BACKGROUND OF THE INVENTION

1. Field of the Invention

The invention relates to a human embryo culture medium, and more particularly to a sequential human embryo culture system comprising a plurality of culture media corresponding to a plurality of patient and embryo groups, and a method for using the culture medium.

2. Background Art

The success of clinical in vitro fertilization (IVF) remains compromised by suboptimal culture conditions, resulting in loss of viability. Therefore, it is of great importance to improve culture conditions as it will improve embryo development in vitro and result in an increase in implantation and pregnancy rates.

Blastocyst culture and transfer is an important technique developed for IVF that maximizes pregnancy rates while minimizing the risk of multiple pregnancy. The ability to grow embryos for 5 or 6 days to the blastocyst stage of development in the laboratory, rather than the traditional 3 days, allows clinicians to determine with greater certainty which embryos should be transferred back to the mother. Consequently, blastocyst culture makes it possible to select the best one or two blastocysts to transfer back to the mother, versus the three or four day-3 embryos that are customarily transferred in order to obtain an acceptable pregnancy rate. Thus, the use of blastocyst transfer reduces the occurrence of potentially risky multiple births.

Blastocyst culture is typically offered only to patients who meet certain restrictive criteria: age <35 (in many programs age <32); at least 15 mature oocytes collected; at least 10 fertilized eggs; at least 6 embryos that have achieved 8-cell stage of division by the third day in culture. The above qualifications lead to a biased selection of only the best candidates. It is not uncommon to lose 60% of those 8-cell embryos when extending the culture another 2 or 3 days.

Three examples of sequential media for embryo development that attempt to take into account the changes in embryo physiology and requirements that occur as the embryo develops from the zygote to the blastocyst are: G1/G2 (Gardner et al., 1998 Hum. Reprod.13, 3434); Universal IVF Medium/M3 (Bertheussen et al., 1997); and PI/Blastocyst Medium (Behr et al., 1998 Am. Soc. Rep. Med. 0-262). Interestingly, medium M3 is a modification of Ham's F-10 and F-12, while Blastocyst Medium is a modification of Ham's F-10). All of these media suffer from the same drawbacks, such that they can be used only with the “best” IVF patients.

The foregoing and other known media are described in Tables 4-7, below, which are taken from Alan O. Trounson and David K. Gardner, eds. Handbook of In Vitro Fertilization, 2d Ed. (CRC Press 1999), at pages 210-212. This and all other references cited herein are incorporated by reference.

### SUMMARY OF THE INVENTION

In almost all IVF centers that do blastocyst transfer, there are selection criteria for deciding which cases can be done as blastocyst transfers vs. day 3 transfers. We have developed a sequential media system designed for culture of human embryos to the blastocyst stage without selection. Even underdeveloped day-3 embryos are successfully brought to the blastocyst stage with our sequential media system. This is the first clinically proven system that offers the possibility of blastocyst transfer for all IVF patients, not merely for the “best” patients.

In prior culture media the sodium lactate, if any, is decreased for the second half of the culture period. The sodium bicarbonate concentration, moreover, may be changed over time in prior art formulations. Thus, in conventional sequential media these concentrations may be permitted to change over the course of embryo development. In addition, conventional media may have higher or lower component ratios or concentrations than those disclosed herein.

Conventional culture media are purchased from one of several vendors and reflect a “one size fits all” approach. The media according to the invention may contain the same standard nutrients and growth factors that are used in conventional human embryo cultures. However, the concentrations of the amino acids, proteins, vitamins and growth factors are selected initially for each patient, based upon her age, level of her FSH and response to ovarian stimulation, and other clinical indications. The changing concentrations in the two media of each pair of media are designed to accommodate the metabolic requirements of the early and more developed embryo.

Further, using this culturing system in conjunction with recently developed blastocyst assisted hatching techniques, e.g. zona drilling, results in high pregnancy (implantation) rates in all age groups without selection.

According to a first aspect of the invention, a sequential human embryo culture system comprises a plurality of culture media corresponding to a plurality of patient and embryo groups, each said medium comprising substances for supporting a human embryo, and a buffer system for maintaining pH substantially constant during embryo development.

According to a second aspect, a sequential human embryo culture system comprises a plurality of culture media corresponding to a plurality of patient and embryo groups, each said medium comprising substances for supporting a human embryo, wherein said supporting substances include sodium lactate.

According to a third aspect, a sequential human embryo culture system comprises a plurality of culture media corresponding to a plurality of patient and embryo groups, each said medium comprising substances for supporting a human embryo, wherein said supporting substances include EDTA.

According to a fourth aspect, a sequential human embryo culture system comprises a plurality of culture media corresponding to a plurality of patient and embryo groups, each said medium comprising substances for supporting a human embryo, wherein said media are substantially phosphate-free.

According to a fifth aspect of the invention, one of the above-described sequential human embryo culture systems is used for supporting a human embryo.

Two sets of sequential media have been designed for human embryo culture to the blastocyst stage. The two media sets are generally age-specific, the first being for patients up to about 32 years of age, and the second being for patients about 32 years of age and older.

Age is not the only criterion for selection of the sequential media system for a specific patient, however. Other factors and clinical indicators such as Day 3 FSH level and previous IVF cycles are considered as well.

In sharp contrast to those prior systems that have a low success rate in bringing embryos to the blastocyst stage, preliminary data using our culture system has demonstrated that over 80% of our patients achieve blastocyst development. Our data from a population of women up to age 44 including those with elevated FSH levels (a poor prognostic indicator) has demonstrated that 54% of day 3 embryos reached the blastocyst stage of development. This has resulted in a 68% pregnancy rate per oocyte retrieval. Among the sub-population of women under the age of 40 with a normal FSH level, the pregnancy rate per oocyte retrieval was 83%.

### DETAILED DESCRIPTION OF EMBODIMENTS OF THE INVENTION

Table 1 shows an example of the components of our first pair of media, which are generally for patients under 32 years of age. Medium KA33-1 is for the first 3 days of embryo development, and medium KA33-2 is for the remaining days.

An example of the second pair of media, usually for older patients, is shown in Table 2. Medium KA33-3 is used for the first three days, for patients 32 years old or older, while medium KA33-4 is used for the remaining days of embryo development.

Table 3 is a summary of Tables 1 and 2.

The pH of culture media formulated for human embryos is conventionally between 7.25 and 7.45+/−0.1. We have found that the pH of the culture media is more important than previously known, in regulating embryo development to the blastocyst stage. It is important for the pH for the media utilized for <32 to be maintained substantially constant at 7.39+/−0.03, and at 7.33+/−0.03 for the ≧32 group.

Commercial media have a wide pH range of +/−0.1 units. As the pH of the media moves further away from the above-noted preferred mean values, embryo development to the blastocyst stage is lost. With our media, at the above—noted mean pH values, we have found that at least 55% of embryos will develop to the blastocyst stage. This number rapidly decreases as the pH increases or decreases. Therefore, care must be taken to avoid fluctuations in the pH of the media. The pH is adjusted by increasing or decreasing the carbon dioxide concentration. The preferred concentration of carbon dioxide has been determined to be in the range of approximately 5.7 to 6.1% by volume in air. Due to incubator variability, each laboratory may make adjustments based on its own carbon dioxide concentration in order to obtain the desired pH.

Our media utilize a bicarbonate/CO₂ buffer system (bicarbonate in the media vs. CO₂ in the atmosphere) to maintain stable pH. The preferred sodium bicarbonate concentration in the media utilized for <32 is about 26.1, while in the media for ≧32 years old the concentration is about 23.0. These concentrations are maintained throughout the full 6 days of the culture period.

Also, in this embodiment substantially constant concentrations of sodium lactate are maintained over the culture period.

The sodium lactate concentrations are given as 10.00 mM and 18.00 mM in Tables 1 and 2, respectively, which represent the best mode now known. However, effective results have been observed for lactate concentrations of 8-13 mM for patients in the <32 group, and 15-23 mM for the ≧32 group.

In addition, the media are phosphate-free. Phosphates have been shown to have a detrimental effect on embryo development in vitro.

The addition of EDTA to the culture media has also been found to have a beneficial effect on embryo development. EDTA is utilized throughout the culturing process for patients ≧32 years of age. However, for <32, the EDTA is only utilized in the medium for the first 3 days, and it is absent in the medium for the next 3 days.

Although the present invention has been described in relation to particular embodiments thereof, many other variations and modifications and other uses will become apparent to those skilled in the art. Therefore, the present invention is not limited by the specific disclosure herein.

TABLE 1
IVF Media Formulation

| | |
|---|---|
| KA33-1 | KA33-2 |

| | |
|---|---|
| Component | Concentration (mM) |

| | | |
|---|---|---|
| Sodium Chloride | 106 | 106 |
| Potassium Chloride | 4.8 | 4.8 |
| Magnesium Sulfate | 0.7 | 0.7 |
| Sodium Bicarbonate | 26.1 | 26.1 |
| EDTA | 0.03 | — |
| Sodium Pyruvate | 0.29 | 0.29 |
| Calcium Chloride | 2.4 | 2.4 |
| Sodium Lactate(L) | 10.0 | 10.0 |
| Taurine | 0.05 | — |
| Glucose | — | 1.1 |
| Alanyl-glutamine | — | 1.1 |
| Arginine | — | 0.18 |
| Alanine | — | 0.05 |
| Asparagine | — | 0.05 |
| L-Aspartic Acid | — | 0.05 |
| Cysteine | — | 0.05 |
| L-Glutamic Acid | — | 0.05 |
| Glycine | — | 0.05 |
| L-Histidine | — | 0.68 |
| Isoleucine | — | 0.10 |
| Leucine | — | 0.10 |
| Lysine | — | 0.12 |
| Methionine | — | 0.03 |
| Phenylalanine | — | 0.05 |
| Proline | — | 0.05 |
| Serine | — | 0.05 |
| Threonine | — | 0.10 |
| L-Typtophan | — | 0.013 |
| Tyrosine | — | 0.05 |
| Valine | — | 0.10 |
| Calcium Pantothenate | — | 0.002 |
| Choline Chloride | — | 0.007 |
| Folic acid | — | 0.002 |
| Myo-Inositol | — | 0.011 |
| Niacinamide | — | 0.008 |
| pyridoxine | — | 0.002 |
| Riboflavin | — | 0.0002 |
| Thiamine | — | 0.003 |
| Human Serum Albumin | 5 mg/ml | 5 mg/ml |
| Gentamicin | 10 ug/ml | 10 ug/ml |

pH = 7.39 +/− 0.03

TABLE 2
IVF Media Formulation

| | |
|---|---|
| KA33-3 | KA33-4 |

| | |
|---|---|
| Component | Concentration (mM) |

| | | |
|---|---|---|
| Sodium Chloride | 98 | 98 |
| Potassium Chloride | 5.0 | 5.0 |
| Magnesium Sulfate | 1.0 | 1.0 |
| Sodium Bicarbonate | 23.0 | 23.0 |
| EDTA | 0.044 | 0.044 |
| Sodium Pyruvate | 0.33 | 0.33 |
| Calcium Chloride | 2.4 | 2.4 |
| Sodium Lactate(L) | 18.0 | 18.0 |
| Taurine | 0.08 | 0.02 |
| Glucose | — | 1.6 |
| Alanyl-glutamine | — | 1.6 |
| Arginine | — | 0.18 |
| Alanine | — | 0.05 |
| Asparagine | — | 0.05 |
| L-Aspartic Acid | — | 0.05 |
| Cysteine | — | 0.05 |
| L-Glutamic Acid | — | 0.05 |
| Glycine | — | 0.05 |
| L-Histidine | — | 0.68 |
| Isoleucine | — | 0.10 |
| Leucine | — | 0.10 |
| Lysine | — | 0.12 |
| Methionine | — | 0.03 |
| Phenylalanine | — | 0.05 |
| Proline | — | 0.05 |
| Serine | — | 0.05 |
| Threonine | — | 0.10 |
| L-Typtophan | — | 0.013 |
| Tyrosine | — | 0.05 |
| Valine | — | 0.10 |
| Calcium Pantothenate | — | 0.002 |
| Choline Chloride | — | 0.007 |
| Folic acid | — | 0.002 |
| Myo-Inositol | — | 0.011 |
| Niacinamide | — | 0.008 |
| pyridoxine | — | 0.002 |
| Riboflavin | — | 0.0002 |
| Thiamine | — | 0.003 |
| Human Serum Albumin | 5 mg/ml | 5 mg/ml |
| Gentamicin | 10 ug/ml | 10 ug/ml |

pH = 7.33 +/− 0.03

TABLE 3

| | Age < 32 | | Age ≧ 32 | |
|---|---|---|---|---|
| | Days 1-3 | Days 4-6 | Days 1-3 | Days 4-6 |
| PH | 7.39 ± 0.03 | 7.39 ± 0.03 | 7.33 ± 0.03 | 7.33 ± 0.03 |
| CO₂ conc. (%) | 5.7-6.1 | 5.7-6.1 | 5.7-6.1 | 5.7-6.1 |
| Sodium | 26.1 | 26.1 | 23.0 | 23.0 |
| bicarbonate | | | | |
| conc. (mM) | | | | |
| Sodium lactate | 10.0 | 10.0 | 18.0 | 18.0 |
| conc. (mM) | | | | |
| EDTA | 0.03 | — | 0.044 | 0.044 |

TABLE 4
Composition (mM) of Simple Salt Solutions with Added Energy
Substrates used in Embryo Culture

| | | Whitten & | | | | | | | Basal | |
|---|---|---|---|---|---|---|---|---|---|---|
| Whitten | Brinster | Biggers | M16 | Earle's* | HTF* | CZB | MTF^{d} | KSOM^{d} | XI HTF* | P1* |
| 1957 | 1965 | 1968 | 1971 | 1971 | 1981 | 1985 | 1990 | 1993 | 1995 | 1998 |

Component

| | | | | | | | | | | | |
|---|---|---|---|---|---|---|---|---|---|---|---|
| NaCl | 118.46 | 119.23 | 68.49 | 94.66 | 116.30 | 101.60 | 81.62 | 114.19 | 95.00 | 97.60 | 101.60 |
| KCl | 4.74 | 4.78 | 4.78 | 4.78 | 5.36 | 4.69 | 4.83 | 4.78 | 2.50 | 4.69 | 4.69 |
| KH₂PO₄ | 1.18 | 1.19 | 1.19 | 1.19 | — | 0.37 | 1.18 | 1.19 | 0.35 | — | — |
| NaH₂PO₄ | — | — | — | — | 1.02 | — | — | — | — | — | — |
| CaCl₂.2H₂O | — | 1.71 | — | 1.71 | 1.80 | 2.04 | 1.70 | 1.71 | 1.71 | 2.04 | 2.04 |
| MgSO₄.7H₂O | 1.18 | 1.19 | 1.19 | 1.19 | 0.81 | 0.20 | 1.18 | 1.19 | 0.20 | 0.20 | 0.20 |
| NaHCO₃ | 24.88 | 25.00 | 25.07 | 25.00 | 26.18 | 25.00 | 25.12 | 25.00 | 25.00 | 25.00 | 25.00 |
| Ca Lactate | 2.54 | — | 1.71 | — | — | — | — | — | — | — | — |
| Na Lactate (D/L) | — | 25.00 | 21.58 | 23.28 | — | 21.40 | 31.30 | 4.79 | 10.00a | 21.4 | 21.4 |
| Na Pyruvate | — | 0.25 | 0.33 | 0.33 | 0.10 | 0.33 | 0.27 | 0.37 | 0.20 | 0.33 | 0.33 |
| Glucose | 5.55 | — | 5.56 | 5.56 | 5.55 | 2.78 | — | 3.40 | 0.20 | — | — |
| BSA (mg/ml) | 1.00 | 1.00 | 4.00 | 4.00 | b | 5.00 | 5.00 | 4.00 | 1.00 | b | c |

Ratios

| | | | | | | | | | | | |
|---|---|---|---|---|---|---|---|---|---|---|---|
| Na/K | 24.21 | 28.39 | 19.34 | 24.00 | 26.79 | 29.31 | 23.01 | 24.18 | 45.68 | 30.77 | 31.63 |
| Ca/Mg | 2.15 | 1.44 | 1.44 | 1.44 | 2.22 | 10.20 | 1.44 | 1.44 | 8.55 | 10.2 | 10.2 |
| L/P | — | 100 | 70.58 | 70.55 | — | 64.85 | 115.93 | 12.95 | 50.00 | 64.85 | 64.85 |

CZB contains 110 μM EDTA, 1.0 mM glutamine, and 5.5 mM glucose after 48 h of culture from the zygote stage.
KSOM contains 10 μM EDTA and 1.0 mM glutamine.
Basal XI HTF contains 100 μM EDTA and 1.0 mM glutamine.
P1 contains 50 μM taurine and 0.5 μM citrate.
*Used in clinical IVF.
^{a}Sodium lactate L-isomer.
^{b}Medium supplemented with serum of human serum albumin.
^{c}Medium supplemented with synthetic serum substitute
^{d}Modifications to these media have included the addition of specific groups of amino acids resulting in significant improvements to mouse zygote development in culture.

TABLE 5
Composition of Ham's F-10 Medium

| | Concentration | | Concentration |
|---|---|---|---|
| Component | (mM) | Component | (mM) |
| NaCl | 126.60 | Leucine | 0.10 |
| KCl | 3.82 | Lysine | 0.20 |
| MgSO₄.7H₂O | 0.62 | Methionine | 0.03 |
| Na₂HPO₄ | 1.31 | Phenylalanine | 0.03 |
| KH₂PO₄ | 0.61 | Proline | 0.10 |
| NaHCO₃ | 14.28 | Serine | 0.10 |
| CaCl₂.2H₂O | 0.30 | Threonine | 0.03 |
| CuSO₄.5H₂O | 0.00001 | Tryptophan | 0.003 |
| FeSO₄.7H₂O | 0.0030 | Tyrosine | 0.12 |
| ZnSO₄.7H₂O | 0.0001 | Valine | 0.03 |
| Phenol Red | 0.034 | Biotin | 0.0001 |
| Sodium Pyruvate | 1.00 | Ca Pantothenate | 0.0015 |
| Calcium Lactate | 1.00 | Choline Chloride | 0.005 |
| Glucose | 6.11 | Cyanocobalamine | 0.001 |
| Alanine | 0.10 | Folic Acid | 0.003 |
| Arginine | 1.21 | Inositol | 0.003 |
| Asparagine | 0.11 | Nicotinamide | 0.005 |
| Aspartic Acid | 0.10 | Pyridoxine | 0.001 |
| Cysteine | 0.26 | Riboflavin | 0.001 |
| Glutamate | 0.1 | Thiamine | 0.003 |
| Glutamine | 1.0 | Hypoxanthine | 0.03 |
| Glycine | 0.1 | Lipoic Acid | 0.001 |
| Histidine | 0.14 | Thymidine | 3.00 |
| Isoleucine | 0.02 | | |

Note:
Penicillin present at 100 U/ml. Streptomycin present at 50 μg/ml. Modifications as per the Center for Reproductive Medicine.

TABLE 6
Composition of a Sequential Medium

| | |
|---|---|
| G1 | G2 |

| | |
|---|---|
| Component | Concentration (mM) |

| | | |
|---|---|---|
| NaCl | 85.16 | 85.16 |
| KCl | 5.5 | 5.5 |
| Na₂HPO₄ | 0.5 | 0.5 |
| MgSO₄.7H₂O | 1.0 | 1.0 |
| CaCl₂.2H₂O | 1.8 | 1.8 |
| NaHCO₃ | 25.0 | 25.0 |
| Sodium Pyruvate | 0.32 | 0.10 |
| Sodium Lactate (L) | 10.5 | 5.87 |
| Glucose | 0.5 | 3.15 |
| Alanine | 0.1 | 0.1 |
| Aspartic Acid | 0.1 | 0.1 |
| Asparagine | 0.1 | 0.1 |
| Arginine | — | 0.6 |
| Cystine | — | 0.1 |
| Glutamate | 0.1 | 0.1 |
| Glutamine | 1.0 | 1.0 |
| Glycine | 0.1 | 0.1 |
| Histidine | — | 0.2 |
| Isoleucine | — | 0.4 |
| Leucine | — | 0.4 |
| Lysine | — | 0.4 |
| Methionine | — | 0.1 |
| Phenylalanine | — | 0.2 |
| Proline | 0.1 | 0.1 |
| Serine | 0.1 | 0.1 |
| Taurine | 0.1 | — |
| Threonine | — | 0.4 |
| Tryptophan | — | 0.5 |
| Tyrosine | — | 0.2 |
| Valine | — | 0.4 |
| EDTA | 0.01 | — |
| HSA | 5 mg/ml | 5 mg/ml |
| Phenol Red | 0.001 g/l | 0.001 g/l |

Note:
Penicillin present at 100 U/ml.

TABLE 7
Concentration (mM) of Ions, Carbohydrates, and Glutamine in
Mammalian Fluids and Embryo Culture Media

| | Human | Human | | Mouse | | | | | | | |
|---|---|---|---|---|---|---|---|---|---|---|---|
| | oviduct | uterine | Human | oviduct | HTF | Ham's | Menezo's | | Basal | | |
| Component | fluid@ | fluid@ | serum | fluid | medium | F-10 | B2/3 | Ksom | XI | G1 | G2 |
| Na | 130 | nd | 145 | 139 | 148 | 143 | 129 | 130.2 | 144.3 | 122.0 | 122.0 |
| Cl | 132 | nd | nd | 165 | 110 | 131 | 114 | 106.4 | 106.4 | 94.3 | 94.3 |
| K | 21.2 | nd | 5.0 | 23.4 | 5.1 | 4.4 | 9.8 | 2.85 | 4.70 | 5.50 | 5.50 |
| Ca | 1.13 | nd | 1.13 | 1.71 | 2.04 | 0.30 | 0.56 | 1.71 | 2.04 | 1.80 | 1.80 |
| Mg | 1.42 | nd | 2.00 | 1.04 | 0.20 | 0.62 | 0.81 | 0.2 | 0.2 | 1.0 | 1.0 |
| S | 12.3 | nd | nd | 8.45 | 0.20 | 0.62 | 0.17 | 0.2 | 0.2 | 1.0 | 1.0 |
| P | 8.69 | nd | nd | 8.93 | 0.37 | 1.92 | 0.90 | 0.35 | — | 0.5 | 0.5 |
| Pyruvate | 0.32 | 0.10 | 0.10 | 0.37 | 0.33 | 1.00 | 2.27 | 0.20 | 0.33 | 0.32 | 0.10 |
| L-Lactate | 10.50 | 5.87 | 0.60 | 4.79 | — | — | — | 10.0 | — | 10.5 | 5.87 |
| D/L-Lactate | — | — | — | — | 21.4 | 2.23 | 0.56 | — | 21.4 | — | — |
| Glucose | 0.50 | 3.15 | 5.00 | 3.40 | 2.78 | 6.11 | 6.67 | 0.2 | 0.00 | 0.50 | 3.15 |
| Glutamine | 0.30 | nd | nd | 0.20 | 0.00 | 0.30 | 0.17 | 1.0 | 1.0 | 1.0 | 1.0 |

Ratios

| | | | | | | | | | | | |
|---|---|---|---|---|---|---|---|---|---|---|---|
| Na/K | 6.1 | — | 29.0 | 5.9 | 29.31 | 32.5 | 13.1 | 45.68 | 30.7 | 22.18 | 22.18 |
| Ca/Mg | 0.80 | — | 0.57 | 1.64 | 10.2 | 0.48 | 0.69 | 8.55 | 10.2 | 1.8 | 1.8 |
| L/P | 32.81 | 58.7 | 6.00 | 12.95 | 64.85 | 2.23 | 0.25 | 50.0 | 64.85 | 32.81 | 58.7 |

@Mid-cycle.

1. A sequential human embryo culture system comprising a plurality of culture media corresponding to a plurality of patient and embryo groups, each said medium comprising substances for supporting a human embryo, and a buffer system for maintaining pH substantially constant during embryo development.

2. The culture system of claim 1, wherein said buffer system comprises sodium bicarbonate.

3. The culture system of claim 1, wherein said buffer system maintains a pH within a range of about ±0.03 units.

4. The culture system of claim 3, wherein said buffer system maintains a pH within a range of about 7.39±0.03 units in a first set of media (Table 1) for patients in a first group, and about 7.33±0.03 units in a second set of media (Table 2) for patients in a second group.

5. The culture system of claim 4, wherein the concentration of sodium bicarbonate is about 26.1 mM in each one of said first set of media and 23.0 mM in each one of said second set of media.

6. The culture system of claim 4, wherein said first and second sets of media are adapted for supporting embryos of generally higher quality and lower quality, respectively.

7. The culture system of claim 1, wherein said supporting substances include sodium lactate.

8. The culture system of claim 7, wherein the concentration of sodium lactate is about 8-13 mM in each one of the first set of media and 15-23 mM in each one of the second set of media.

9. The culture system of claim 8, wherein the concentration of sodium lactate is about 10.0 mM in each one of the first set of media and about 18.0 mM in each one of the second set of media.

10. The culture system of claim 1, wherein said supporting substances include EDTA.

11. The culture system of claim 1, wherein said media are substantially phosphate-free.

12. A sequential human embryo culture system comprising a plurality of culture media corresponding to a plurality of patient and embryo groups, each said medium comprising substances for supporting a human embryo, wherein said supporting substances include sodium lactate.

13. The culture system of claim 12, wherein the concentration of sodium lactate is about 8-13 mM in each one of a first set of media and 15-23 mM in each one of a second set of media.

14. The culture system claim 13, wherein the concentration of sodium lactate is about 10.0 mM in each one of the first set of media and about 18.0 mM in each one of the second set of media.

15. The culture system of claim 12, wherein said supporting substances include EDTA.

16. The culture system of claim 12, wherein said media are substantially phosphate-free.

17. A sequential human embryo culture system comprising a plurality of culture media corresponding to a plurality of patient and embryo groups, each said medium comprising substances for supporting a human embryo, wherein said supporting substances include EDTA.

18. The culture system of claim 17, wherein:
in a first set of media, the EDTA concentration is about 0.03 in a first medium and is substantially zero in a second medium; and
in a second set of media, the EDTA concentration is about 0.044 in both a first medium and a second medium.

19. A sequential human embryo culture system comprising a plurality of culture media corresponding to a plurality of patient and embryo groups, each said medium comprising substances for supporting a human embryo, wherein said media are substantially phosphate-free.

20. A method of supporting a human embryo, comprising the use of a sequential human embryo culture system comprising a plurality of culture media corresponding to a plurality of patient and embryo groups, according to claim 1.

21. The method of claim 20, further comprising the step of selecting either the first set or the second set of media for a given patient on the basis of at least one clinical indicator for said patient, said at least one indicator being selected from the group consisting of: her age, her FSH level, and her previous IVF cycles.

22. The method of claim 21, wherein either the first or the second set of media is selected for a given patient based on her age.

23. The method of claim 21, wherein a first medium of the selected set of media is used for the first three days of in vitro embryo development and a second medium from the selected set of media is used for the remaining days of in vitro embryo development.
